(19) United States
(12) Patent Application Publication
(10) Pub. No.: US 2006/0099247 A1
(43) Pub. Date: May 11, 2006

(54) Liquid, gas and/or vapor phase delivery systems

(75) Inventors: Evelyna D. Cantwell, Fort Lauderdale, FL (US); John W. Cantwell, Fort Lauderdale, FL (US); Efim Ya. Lyublinski, Solon, OH (US); Yefim Vaks, South Euclid, OH (US)

Correspondence Address: BENESCH, FRIEDLANDER, COPLAN & ARONOFF LLP;ATTN: IP DEPARTMENT DOCKET CLERK, 2300 BP TOWER, 200 PUBLIC SQUARE, CLEVELAND, OH, 44114, US

(73) Assignee: Byrd-Walsh, LLC.

(21) Appl. No.: 10/986,101

(22) Filed: Nov. 10, 2004

(51) Int. Cl. A61K 9/48; A61K 9/46; A61M 11/00

(52) U.S. Cl. 424/451; 424/466; 128/200.23

(57) ABSTRACT

This invention relates generally to liquid, gas and/or vapor phase delivery systems and, more particularly, to delivery systems that incorporate at least one layer, film and/or capsule that produces a liquid, gas and/or vapor phase compound or compounds for use therein.

### FIELD OF THE INVENTION

This invention relates generally to liquid, gas and/or vapor phase delivery systems and, more particularly, to delivery systems that incorporate at least one layer, film and/or capsule that produces a liquid, gas and/or vapor phase compound or compounds for use therein.

### BACKGROUND OF THE INVENTION

The selective generation of, or the selective increase in a partial pressure of, one or more gaseous products, or the delivery of one or more liquid and/or vapor phase compounds, can be advantageous in a wide variety of applications. For example, in the anti-corrosion realm, the addition of a volatile corrosion inhibitor to the atmosphere can significantly reduce or halt surface corrosion on the item(s) placed in the enclosure, regardless of whether or not the enclosure itself is airtight.

In the realm of decontamination and/or sterilization, it is sometimes desirable to increase the atmospheric concentration of oxygen. For example, raising oxygen levels fights infections, or the possibility that an infection will result, by killing bacteria (many of which cannot survive in an oxygen-rich environment) and suppressing their deadly toxins. Oxygen also creates “free radicals,” unstable oxygen molecules that are lethal to germs. Oxygen can also stimulate roving immune cells called phagocytes, which scavenge for infectious microbes.

An elevation in the oxygen concentration surrounding a wound also helps heal injuries by fostering the growth of tiny blood vessels and/or capillaries that funnel in important nutrients and by keeping existing blood vessels open. Oxygen is also conducive to the production of collagen, the main wound-repairing connective tissue in the body. By speeding up healing processes, oxygen may also aid in the regeneration of nerve cells.

With regard to the introduction of a compound in a liquid and/or vapor phase, an increased amount of water or water vapor, for example, can be beneficial in a wide variety of instances, such as the preservation of cut flowers or potted plants. Additionally, a wide variety of medicines, pharmaceuticals or other therapies are administered as liquids or in a gas or vapor phase in order to facilitate introduction of the desired treatment to a desired portion of an individual's body (e.g., the lungs, skin, eyes, etc.).

### DRAWINGS

In the accompanying drawings, liquid, gas and/or vapor-phase producing layers are illustrated that, together with the detailed description provided below, describe example embodiments of the method. It will be appreciated that the illustrated boundaries of elements in the drawings represent one example of the boundaries. One of skill in the art will appreciate that one element may be designed as multiple elements or that multiple elements may be designed as a single element. An element shown as an internal component of another element may be implemented as an external component and vice-versa.

In the drawings and description that follows, like elements are identified with the same reference numerals.

The drawings are not to scale and the proportion of certain elements may be exaggerated for the purpose of illustration.

FIG. 1 is a cross-sectional view of a liquid, gas and/or vapor phase compound producing layer, according to one embodiment of the present invention.

FIG. 2 is a schematic view of a unit which can be used to produce microspheres and/or microcapsules according to one method of the present invention.

FIGS. 3 through 14 are cross-sectional views of a liquid, gas and/or vapor phase compound producing layer according to different embodiments of the present invention.

FIG. 15A is a perspective view of a film that incorporates a liquid, gas and/or vapor phase compound producing layer according to the embodiment of FIG. 3.

FIG. 15B is a perspective view of a film that incorporates a liquid, gas and/or vapor phase compound producing layer according to another embodiment of the present invention.

FIGS. 16 through 18 are perspective views of additional embodiments of films that incorporate at least one liquid, gas and/or vapor phase compound producing layer according to the embodiment of FIG. 3.

FIG. 19 is a cross-sectional view of a liquid, gas and/or vapor phase compound producing capsule according to one embodiment of the present invention, that incorporates therein a film according to the embodiment of FIG. 15.

FIG. 20 is a cross-sectional view of a liquid, gas and/or vapor phase compound producing capsule according to one embodiment of the present invention, that incorporates therein a film according to the embodiment of FIG. 16.

FIG. 21 is a cross-sectional view of a liquid, gas and/or vapor phase compound producing capsule according to another embodiment of the present invention.

FIG. 22 is a cross-sectional view of a liquid, gas and/or vapor phase compound producing capsule according to another embodiment of the present invention.

### DETAILED DESCRIPTION

This application describes liquid, gas and/or vapor phase delivery systems that enable the delivery of one or more liquid, gas, or vapor compounds, or any combination thereof, to a specified location, enclosure or localized area in an inexpensive, compact and/or light-weight manner. The ability to deliver a compound in a liquid, gas or vapor form has numerous advantages in a wide variety of technical fields including, but not limited to, medicine, corrosion protection, waste disposal, food storage, food and beverage packaging, cosmetics, pharmaceuticals, and horticulture.

For example, the ability to deliver a pharmaceutical compound as a liquid, gas and/or vapor via an inexpensive, compact and/or light-weight manner, is advantageous in that it can enable the administration of certain drugs or wound healing promoters in both traditional settings (e.g., hospitals, doctors' offices, emergency rooms, trauma centers, etc.) and non-traditional settings (e.g., on the battlefield, in the backcountry, at an accident site, etc.).

The following includes definitions of selected terms employed herein. The definitions include various examples and/or forms of components that fall within the scope of a term and that may be used for implementation. The examples are not intended to be limiting. Both singular and plural forms of terms may be within the definitions.

“Degradable polymer,” as used herein, refers to any polymer that breaks down via a method that accelerates the decomposition of the polymer (e.g., photodegradation or biodegradation).

“Biodegradable polymer,” as used herein, refers to any polymer that is consumed and/or broken down by microorganisms such as bacteria, fungi, and/or algae.

“Block polymer,” as used herein, refers to a high polymer whose molecule is composed of alternating sections of one chemical composition separated by sections of a different chemical composition or by a coupling group of low molecular weight. An example of a block polymer is blocks of polyvinyl chloride interspersed with blocks of polyvinyl acetate.

“Crosslinkable polymer composition,” as used herein, refers to any polymer composition that containing functional crosslinkable groups (e.g., carboxy-, hydroxy-) or amino groups (e.g., polysaccharide or polypeptide groups) that can be crosslinked via the use of a crosslinking agent.

“Film” and “films,” as used herein, refer to any sheet of material, regardless of construction, having a thickness of less than about 2 inches. This definition includes films having one or more polymer layers, paper layers, metal layers, or any combination thereof.

“Microspheres,” as used herein, refers to granules made from one material or a homogeneous mixture of materials that, in one embodiment, are substantially spherical. As discussed below, microspheres in accordance with the present invention are not limited to solely spherical shapes.

“Microcapsules,” as used herein, refers to granules having a solid shell and a liquid, powder, or solid core that, in one embodiment, are substantially spherical,. As discussed below, microcapsules in accordance with the present invention are not limited to solely spherical shapes.

“Typical atmospheric conditions,” as used herein, refers to conditions present when the temperature is in the range of about −40° C. to about 120° C., an atmospheric pressure of about 870 millibars (mb) to about 1100 mb, and a relative humidity of 0% to 100%.

“Vapor phase,” as used herein, refers to the state of a substance that exists below its critical temperature, and that may be liquefied by application of sufficient pressure, or the gaseous state of a substance that is a liquid under typical atmospheric conditions produced via a vaporization technique (e.g., via an atomization and/or misting nozzle).

“Volatile corrosion inhibitor,” as used herein, refers to a volatile inhibiting compound or a mixture of compounds with a finite vapor pressure that, under a given set of conditions, can generate vapors that may or may not condense on any surface the vapors come into contact with.

Additionally, it should be noted that in the following text, range limits may be combined.

Liquid, Gas and/or Vapor Phase Producing Layers

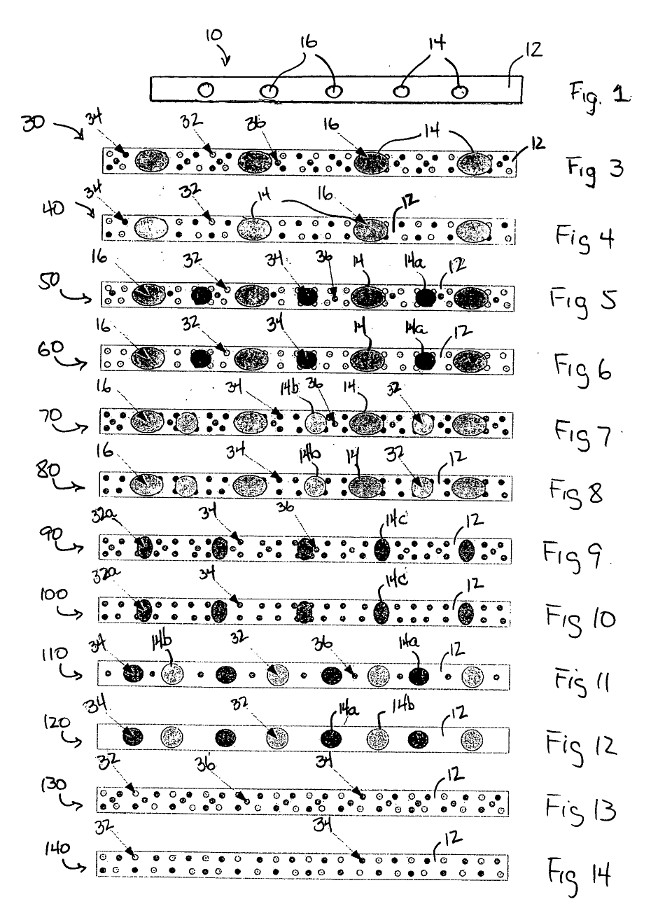

Referring now to FIG. 1, one embodiment of a liquid, gas and/or vapor phase compound producing layer is disclosed. Layer 10 comprises microspheres and/or microcapsules 14 that are contained or embedded in a suitable material 12. Microspheres and/or microcapsules 14 contain therein at least one compound 16 that can produce a liquid, gas and/or vapor phase compound under typical atmospheric conditions. As will be discussed in detail below, microspheres/microcapsules 14 can be formed from a wide variety of compounds so long as the compound used to form microspheres/microcapsules 14 permits the containment and eventual release of compound 16. Although FIG. 1 illustrates compound 16 as being discretely contained within one type of microcapsule 14, other configurations are possible. For example, compound 16 could be mixed with the material used to produce microspheres and/or microcapsules 14 to yield a homogenous mixture thereof. This homogenous mixture could then be used to form the desired microspheres 14, as is discussed in detail below. In another embodiment, microspheres and/or microcapsules containing two or more compounds could be present in material 12.

Compounds that can be used to form material 12 include, but not limited to, polymers, co-polymers, terpolymers, block polymers, block co-polymers, open-celled foams, closed-cell foams, paper, cellulose, adhesives, and gels.

In one embodiment, material 12 is a polymer, co-polymer, terpolymer, or block co-polymer layer. Suitable polymers for use as material 12 include, but are not limited to, polyolefins, polyethylenes, polystyrenes, polypropylenes, polyurethanes, polymethacrylates, degradable polymers, biodegradable polymers, starch-based polymers, polyvinyl alcohols, polyvinyl acetates, polyenlketones, or co-polymer combinations of two or more thereof.

As noted above, biodegradation is defined as a process carried out by microbes; e.g., bacteria, fungi, algae, wherein a polymer chain is cleaved via enzymatic activity to form monomers or short chains. Microbes generally assimilate the monomers or short chains. For example, in an aerobic environment, these monomers or short chains are ultimately oxidized to carbon dioxide, water, and new cell biomass. In an anaerobic environment, the monomers or short chains are ultimately transformed into carbon dioxide, water, acetate, methane, and cell biomass. Efficient biodegradation requires that direct physical contact be established between the biodegradable material and the active microbial population or the enzymes produced by the active microbial population.

Many biodegradable polymers have been developed and are useful as material 12. They include, but are not limited to, cellulose or cellulose derivatives having a low degree of substitution—which is biodegradable under certain conditions. Additional useful biodegradable polymers include, but are not limited to, polyhydroxyalkanoates (PHA), such as polyhydroxybutyrate (PHB), linear ε-polycaprolactone (PCL), or copolymers of polyhydroxybutyrate and polyhydroxyvalerate (PHBV), polylatic acid polymers, polyglycolic acid polymers, biodegradable polyester amide polymers, biodegradable polyester urethane polymers and biodegradable copolymers of any combination of two or more of the above. Such copolymers could include two or more of the same type of polymer, for example, two or more different biodegradable polyesters.

In another embodiment, the compound used for material 12 is an open or closed-cell foam. Suitable compositions that may be used to produce foams (either open-cell, closed-cell, or both) include, but are not limited to, acrylonitrile butadiene styrene (ABS), polyvinyl chlorides (PVCs), polyurethanes, polypropylenes, crosslinkable polymer compositions, polystyrenes, polyethylenes, polyolefins, and co-polymers of at least two polyolefins.

In one embodiment, the compound used for material 12 is either gas-permeable or liquid-permeable. In another embodiment, the compound used for material 12 is gas-permeable and liquid-permeable. In yet another embodiment, the compound used for material 12 is both gas-permeable and water-permeable.

Turning to the microspheres and/or microcapsules 14, the differences between these two products and exemplary production methods for both will be discussed below. It should be noted that the claimed invention can utilize solely microspheres, solely microcapsules, or a combination of both. Additionally, both microspheres and microcapsules can be formed from almost any material that can be liquefied and solidified again afterwards. Furthermore, although FIG. 1 illustrates microspheres/microcapsules 14 that are spherical in shape, the present invention is not limited thereto. Other suitable shapes for microspheres/microcapsules 14 include, but are not limited to, elliptical, oval, tear drop-shaped, and barbell-shaped.

As noted above, the compound used to form the microspheres and/or microcapsules of the present invention should be chosen so as to contain, or encapsulate, and eventually permit the release of compound 16 contained therein (be it discretely or homogenously). In other words, the compound used to form the microspheres and/or microcapsules of the present invention should not be soluble or subject to short term degradation by the compound contained within the microspheres and/or microcapsules. Short term degradation is defined as any unwanted release of compound 16 due to the degradation of the compound used to form microspheres and/or microcapsules 14. In other words, and depending upon the exact nature of compound 16 and the shelf-life sought for the products containing the liquid, gas and/or vapor phase delivery systems of the present invention, the compound used to form microspheres and/or microcapsules 14 should resist degradation from compound 16, maintain the effectiveness of compound 16, and/or prevent the release of compound 16 for at least about 3 months, at least about 6 months, at least about 12 months, or even at least about 18 months under typical atmospheric conditions. In another embodiment, the compound used to form microspheres and/or microcapsules 14 should resist degradation from compound 16, maintain the effectiveness of compound 16, and/or prevent the release of compound 16 for at least about 2 years, at least about 3 years, at least about 4 years, or even at least about 5 years, under typical atmospheric conditions.

The compound or compounds used to form microspheres and/or microcapsules include, but are not limited to, waxes that are suitable for controlled release applications, hydrocolloids, polymers such as polyethylenes, polypropylenes, polymethacrylates, polyesters, polyurethanes, polyurethane/polyurea co-polymers, polyureas, polyethersulfones, other thermoplastic polymers, or the non-curing components of thermosetting polymers like novolak and epoxy resins, or combinations thereof. Inorganic materials can also be used to generate microspheres and/or microcapsules. For example, inorganic oxides including, but not limited to, ZrO₂, HfO₂, SiO₂, Al₂O₃, and ZrHfO₄ can be used to produce microspheres and/or microcapsules.

Matrix encapsulation, which yields microspheres, is desirable if the active agent is to be distributed homogeneously in the compound used to form the microspheres. In one embodiment, microspheres are spherical particles formed from a compound or a homogenous mixture of compounds. Microspheres of this type are able to release the encapsulated active agents over a defined time.

Microencapsulation is defined as the embedding of at least one ingredient (e.g., active agent or compound 16) into at least one other compound used to form the shell of microcapsules 14. The active agent/compound may not be suitable to use independently due to one or more reasons such as low solubility, reactivity (too low or high), low stability under certain conditions. Alternatively, none of the above concerns may be present. Rather, it may be desirable to optimize the properties of the active agents by, for example, administering the desired active agent via controlled release over time or controlled release over a localized area.

Microencapsulation techniques can produce a variety of different microcapsules. For instance, microcapsules can be produced with a solid shell and a homogeneous mixture as the core (regardless of whether the homogenous mixture is a liquid, solid, gas, or powder). In another embodiment, microcapsules with solid shell and a suspension as the core can be produced. A drip casting process can be used to produce microcapsules with a solid shell and a discretely contained core within the solid shell.

The liquefaction of the starting material to be used in forming the microspheres, the shells of the microcapsules, and/or the cores of the microcapsules can be achieved by different means including, but not limited to: (1) dissolving the raw material in a suitable solvent (this process is called a binder process because a temporary binder is often used); (2) melting of the raw material in a melting furnace (melting process); (3) generating a gel from a solution (SOL-GEL process); or (4) a fusion process. Microspheres and/or microcapsules can be produced using one or more of the above-mentioned methods, or other methods known to those of skill in the art. Regarding the above-mentioned methods, each process will in turn be explained in more detail below.

Exemplary Microsphere/Microcapsule Production Processes

Binder Process

One of the commonly used processes for the production of microspheres and/or microcapsules is based on the use of a temporary binder which agglutinates the particles of the starting material. Often, but not always, the binder used is an organic or inorganic powder. A binder is generally used for the production of microspheres and/or microcapsules made from inorganic oxides like ZrO₂, HfO₂, SiO₂, Al₂O₃, and ZrHfO₄, but may be used for microspheres/microcapsules made from polymer compounds. The binder is usually an organic substance like alginate, gelatin, agar-agar, cellulose or an inorganic solid such as SiO₂. The organic or inorganic binders are removed from the resulting microspheres and/or microcapsules by a slag process with temperatures of about 300° C.

The binder process is primarily suited for manufacturing microspheres, as the binder process lends itself to the production of homogenous particles. However, this is not meant to preclude the possibility that such a process could be used to produce microcapsules.

Melting Process

The production of microspheres and/or microcapsules by a melting process is based on a change of state (solid to liquid) of the starting material due to a temperature increase. Accordingly, the process can only be applied to materials that can be melted and that have a sufficiently low viscosity. Viscosities of about 10 mPa·s are optimal but nearly any material with a viscosity up to about 200 mPa·s can be processed. Depending on the properties of the starting material it is also possible to process materials with viscosities up to about 10,000 mpa·s.

It should be noted that the viscosity of the starting material has a direct correlation to the size of the microspheres produced. That is, the lower the viscosity of the starting material, the smaller the diameter of microspheres that can be produced by the melting process. Thus, the ability to make small diameter microspheres becomes harder, if not impossible, as the viscosity of the starting material increases. The temperature used to produce the liquid starting material for the production of microspheres by a melting process is limited only by the chemical nature of starting material itself. Any temperature that produces a liquid compound can be used. Accordingly, it is even possible to process pure silicon at about 1450° C.

SOL-GEL Process

The production of microspheres and/or microcapsules by a SOL-GEL process is based on the formation of a gel from a solution containing the compound from which the microspheres and/or microcapsules are to be produced. As an example, if a metal ion has an oxide compound with a low solubility, the oxide compound can be precipitated from a pre-neutralized solution by the addition of a base. In this type of reaction, a metal ion hydroxide is formed which passes over into the oxide. In the meantime, the viscosity of the solution increases. Therefore, this process results in the formation of a gel from a solution. The SOL-GEL process is used for the production of extraordinarily pure oxide microspheres (e.g., for the production of Al₂O₃ microspheres and/or microcapsules).

Microspheres produced in accordance with the SOL-GEL process are precipitated from the solution. Accordingly, as a general rule, the need for a binder is eliminated. Therefore, it is possible to produce extraordinarily pure microspheres with a very large surface areas. The SOL-GEL process can also be applied to the production of highly pure or uncontaminated microspheres for use as, for example, catalysts, catalyst supporters and other high-purity compounds.

Fusion Process

The production of microspheres and/or microcapsules by a fusion process is based on a change of state (solid to liquid) of the starting material by temperature and then subjecting the liquid starting material to a vibrating nozzle system. Depending upon the design of the nozzle system, the fusion process can be used to produced microspheres or microcapsules.

As noted above, the fusion process relies on a liquid starting material. Generally, the starting material is a liquid plastic that is pumped through a vibrating nozzle system where upon exiting the fluid stream breaks up into uniform droplets. The surface tension of these droplets molds them into perfect spheres in which solidification is induced during a short period of free fall. Solidification can be induced in a gaseous medium through cooling or drying and/or in a liquid medium through cooling or chemical reaction. The amplitude and frequency of the nozzle oscillation or the liquid oscillation are held constant to attain a monodisperse grain size distribution. However, these parameters can be changed during production to yield microspheres and/or microcapsules with any desired grain size distribution.

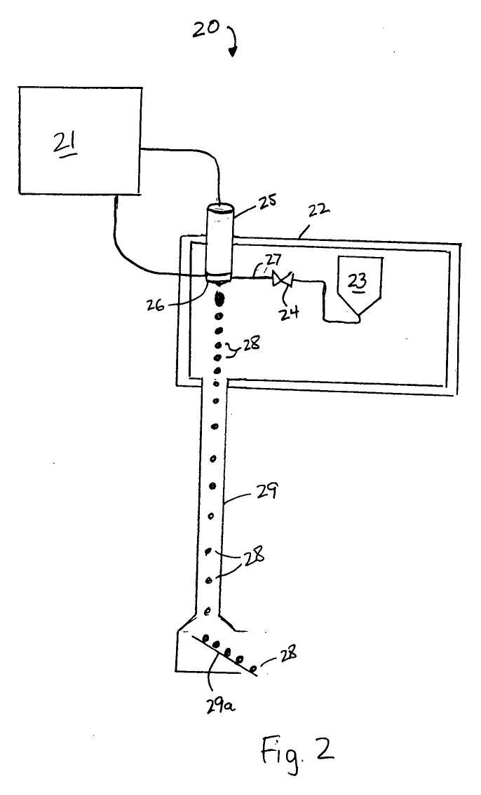

Referring now to FIG. 2, FIG. 2 illustrates an example of a microsphere/microcapsule production device 20. Device 20 has a control cabinet 21 that is connected to a heating cabinet 22. Control cabinet 21 is designed to provide control of the elements that are contained within heating cabinet 22, as well as the remaining elements of device 20. Heating cabinet 22 comprises a feed tank 23, a pressure control valve 24, a vibrator unit 25, and a nozzle 26. Feed tank 23, which is connected to nozzle 26 via pressure control valve 24 and supply line 27, contains therein a supply of starting material. In one embodiment, the starting material contained within supply tank 23 is supplied as a solid that liquefies pon being heated within heating cabinet 22. In another embodiment, supply tank 23 can be filled with an initial supply of liquid starting material that is maintained in a liquid state due to the heat supplied from heating cabinet 23. In yet another embodiment, a continuous supply of liquid or solid starting material can be supplied to supply tank 23 from an external source (not shown). Pressure control valve 24, in conjunction with control cabinet 21, controls the pressure of the liquid starting material supplied to vibrator unit 25.and nozzle 26. Once the liquid starting material 23 reaches nozzle 26 of vibrator unit 25, the vibration of nozzle 26 causes the liquid starting material to break-up into perfect spherical droplets 28. Depending on the type of nozzle 26 used either microspheres or microcapsules are produced.

For the production of microspheres, a single nozzle design is used for nozzle 26. Such a nozzle design provides for the production of microspheres formed of one material or a homogenous mixture of at least two materials. For the production of microcapsules, a dual nozzle design is used where nozzle 26 produces two concentric droplets. In this embodiment, the material used for the inside of the microcapsule can be chosen independently from the material used for the shell of the microcapsule. Any material can be used for the core of the microcapsules, so long as the core material does not structurally weaken the shell material and the material to be used in the core is either a gas or liquid, or can be gasified or liquefied under the conditions used to produce the shells of the microcapsules. In the case where microcapsules are to be produced, device 20 can further include a second supply line (with or without a pressure valve) to supply core material to nozzle 26. Upon completion of the microcapsule fusion formation process, depending upon the nature of the core material, the cores of the microcapsules produced thereby can be either gas, liquid or solid.

In still another embodiment, nozzle 26 can be designed to produce three or more concentric droplets, thereby permitting the production of microcapsules with a shell and at least two inner concentric layers. In yet another embodiment, nozzle 26 can be designed to produce microspheres or microcapsules having non-spherical shapes such as, but not limited to, elliptical, oval, tear drop-shaped, and barbell-shaped.

As was noted above, the amplitude and frequency of the nozzle oscillation or the liquid oscillation are held constant to attain a monodisperse grain size distribution. However, these parameters can be changed during production to yield microspheres and/or microcapsules with any desired grain size distribution.

Once the formation of spherical droplets 28 is complete, spherical droplets 28 pass into cooling chamber 29. As noted above, solidification of the microspheres or the shells of the microcapsules can be induced in a gaseous medium through cooling or drying and/or in a liquid medium through cooling or chemical reaction. In the case of microcapsules, depending upon the nature of the core material, the cooling process may render the core of the microcapsules solid. Alternatively, the core of the microcapsules can remain in a liquid and/or gas state even after cooling of the shell material is complete.

To ensure that spherical droplets 28 are not flattened on entry into cooling chamber 29 (i.e., undergo a geometric change), an accurate angular catch 29a is provided at the bottom end of cooling chamber 29. Alternatively, catch 29a can be replaced by a liquid layer, so long as the liquid used has a density greater than the liquid, if any, used in cooling chamber 29.

Microsphere production units identical or similar to device 20 can be designed and constructed from laboratory scale up to full size production plants. Such units are available from Brace GmbH of Germany. Based on a sphere diameter of 1 mm, lab installations typically have a plastic throughput of about 20 kg/h, pilot plants about 100 to 200 kg/h, and production units can be installed that process up to 2 metric tons/h.

In addition to the use of device 20 in conjunction with the above described fusion process, device 20 or a similar device with nozzle configurations described above could be designed to work in conjunction with any of the other microsphere/microcapsule production processes described above.

Any of the above processes can be designed to produce microspheres/microcapsules with a diameter of about 10 microns (μm) to about 20 millimeter (mm). In another embodiment, the diameter of the microspheres and/or microcapsules produced for use in the present invention range from about 100 μm to about 10 mm, or even from about 250 μm to about 1 mm. As would be apparent to those of skill in the art, the size of microspheres and/or microcapsules 14 used in conjunction with the present invention is not critical, and any sized microsphere and/or microcapsule can be used so long as microspheres and/or microcapsules 14 are suitable for incorporation and/or embedding into material 12. Accordingly, the intended use for layer 10 and/or the thickness of material 12 will generally dictate the size necessary for microspheres and/or microcapsules 14.

As noted above, in the embodiment where layer 10 contains microcapsules, microcapsules 14 can contain therein a discrete inner core of compound 16. Once again, compound 16 can be any compound (or combination of compounds) that can produce a liquid, gas and/or vapor phase compound under typical atmospheric conditions. Depending upon the nature of compound 16, layer 10 may not be able to produce a liquid, gas and/or vapor throughout the complete temperature range discussed above in connection with typical atmospheric conditions. In such cases, the operating conditions for layer 10 will be noted on the packing for the product containing layer 10, or by some other equivalent means.

Compound 16 can be, but is not limited to, one or more oxygen gas-producing compounds (e.g., hydrogen peroxide, lithium perchlorate, sodium perchlorate, potassium perchlorate, lithium peroxide, sodium peroxide, potassium peroxide, calcium peroxide, magnesium peroxide, barium peroxide, lead peroxide, carbamide peroxide (CH₆N₂O₃), potassium nitrate, potassium permanganate, chromium (VI) oxide, potassium dichromate, etc.), one or more nitrogen gas-producing compounds, one or more vapor phase corrosion inhibiting compounds, water, one or more anti-bacterial compounds, one or more anti-viral compounds, one or more anti-static compounds, one or more disinfectants, one or more pain-reliving compounds (e.g., ibuprofen, acetaminophen, naproxen sodium, etc.), one or more anti-coagulant compounds, one or more blood-thinning compounds (e.g., heparin), one or more blood clotting compounds/promoters, one or more fragrance compounds, one or more stimulants (e.g., stimulants to increase blood flow, energy level, etc.), one or more vitamins, one or more amino-acid supplements, one or more skin-care products, one or more compounds designed to treat acne, one or more odor suppressants, one or more odor enhancing compounds, one or more pharmaceutical compounds, one or more UV-protectant compounds, one or more lubricant compounds, one or more fertilizers, one or more polishing compounds, one or more cleaning compounds, one or more flavor compounds, one or more citrus extracts, one or more medicinal compounds, or compatible mixtures of two or more different types of compounds.

Suitable volatile corrosion inhibitors are disclosed in U.S. Pat. Nos. 4,290,912; 5,320,778; and 5,855,975, and are incorporated herein by reference in their entirety for their teachings of such compounds. For example, useful volatile corrosion inhibitors include, but are not limited to, anhydrous sodium molybdate and mixtures of such molybdates with sodium nitrite, benzotriazole, and mixtures of benzoates of amine salts with benzotriazole, nitrates of amine salts, and C₁₃H₂₆O₂N.

In another embodiment, compound 16 can be a gas. Suitable gases for inclusion in the interior of microcapsules include, but are not limited to, oxygen, nitrogen, pain-reliving gases (e.g., nitrous oxide). As would be apparent to one of skill in the art, taking into consideration the chemical compound being used to form the microcapsule shells, any gas or gases to be included in the interior of the microcapsules need to be non-reactive, non-combustible, and/or non-explosive at the processing parameters necessary to form the microcapsules shells.

In operation, layer 10 of FIG. 1 can produce a liquid, gas and/or vapor phase compound via a number of different methods. For example, if material 12 is liquid-permeable and the microspheres and/or microcapsules contained therein are able to broken down by a liquid, layer 10 can produce a liquid, gas and/or vapor phase composition upon exposure to a liquid which breaks down the compound from which the microspheres are formed and/or breaks down the shells of the microcapsules. Alternatively, if material 12 is only gas-permeable, the release of compound 16-may be caused by the presence of a gas that breaks down or decomposes the microspheres and/or the shells of the microcapsules contained in material 12.

Once compound 16 has been released from microspheres/microcapsules 14, the process of generating the desired liquid, gas and/or vapor phase composition can include a chemical reaction, a phase change and/or be the result of the physical release of compound 16 from microspheres and/or microcapsules 14. In the case where the release of the desired liquid, gas and/or vapor is the result of a chemical reaction, the chemical reaction can occur between compound 16 and the liquid used to breakdown or dissolve the shells of microcapsules 14. Alternatively, compound 16 can react with the ambient atmosphere present in material 12 in which microcapsules 14 are located. In yet another embodiment, compound 16 can react with one or more compounds also present in material 12, regardless of whether the additional compounds are contained in their own set of microspheres/microcapsules or are just embedded in material 12.

In another embodiment, or in addition to the above-embodiment, in the case where microcapsules 14 are present in layer 10, layer 10 can produce a liquid, gas and/or vapor phase compound upon pressure being applied to either one side or both side of material 12, causing microcapsules 14 to burst and release the compound 16 contained therein. In this instance, it is only necessary for material 12 to be liquid-permeable if a liquid compound 16 is being used in microcapsules 14.

Gas, Liquid and/or Vapor Phase Producing Layers of FIGS. 3 through 14

Referring now to FIGS. 3 through 14, additional embodiments of a liquid, gas and/or vapor phase compound producing layers 30, 40, 50, 60, 70, 80, 90, 100, 110, 120, 130 and 140 are disclosed. In the embodiments of FIGS. 3 through 14, the reaction to produce the desired liquid, gas and/or vapor phase compound relies upon the occurrence of a chemical reaction. However, the present invention and the embodiments of FIGS. 3 through 14 are not limited thereto. The embodiments disclosed in FIGS. 3 through 14 will discussed in relation to the production of oxygen gas. However, it should be noted that the embodiments of FIG. 3 through 14 are not limited to only to an oxygen gas producing configuration. As would be apparent to one of skill in the art, the embodiments of FIG. 3 through 14 can be used to produce any desired liquid, gas and/or vapor phase compound regardless of whether or not a chemical reaction or a catalyzed chemical reaction occurs. In one embodiment, no chemical reaction occurs in or in the area surrounding material 12. Instead, compound 16 is released from layer 10 in the desired phase, and exits material 12 in order to be delivered as desired.

Referring specifically to FIG. 3, layer 30 of FIG. 3 comprises microcapsules 14 that are incorporated in a gas-permeable material 12. Microcapsules 14 contain water 16 therein, but as noted above can contain a wide variety of other liquid, gas and/or vapor phase producing compounds. In additional, material 12 contains or has embedded therein, in solid or powder form, an oxygen-releasing compound 32, a catalyst compound 34 and a second active compound 36.

The oxygen-releasing compound 32 is selected from any compound that can produce oxygen gas via a catalyzed reaction, decomposition, or a heat-driven reaction. Such compounds include, but are not limited to, lithium perchlorate, sodium perchlorate, potassium perchlorate, lithium peroxide, sodium peroxide, potassium peroxide, calcium peroxide, magnesium peroxide, barium peroxide, lead peroxide, carbamide peroxide (CH₆N₂O₃), potassium nitrate, potassium permanganate, chromium (VI) oxide, potassium dichromate, and mixtures of two or more thereof.

The catalyst compound 34 is selected from any catalyst compound that can catalyze the production of oxygen gas from the oxygen-releasing compound 32. Such compounds include, but are not limited to, sodium permanganate, potassium permanganate, and manganese (IV) oxide. In another embodiment, catalyst compound 34 can be eliminated if the oxygen-releasing compound 32 yields oxygen via a decomposition reaction (e.g., hydrogen peroxide) or via a heat driven reaction (e.g., barium peroxide, lead peroxide, carbamide peroxide (CH₆N₂O₃), potassium nitrate, potassium permanganate, chromium (VI) oxide, or potassium dichromate).

The second active compound 36 can be, for example, a blood thinning compound, an anti-coagulant, a pain relieving compound, a blood-clotting compound, an anti-bacterial compound, an anti-viral compound, or a pharmaceutical compound. As would be apparent to one of skill in the art, the embodiment of FIG. 3 is not limited to one second active compound or the second active compounds listed above. Rather, any one or more compounds that can be used as compound 16 can be used as additional active compounds, and can be embedded or deposited in material 12.

Alternatively, any one or all of oxygen-releasing compound 32, catalyst compound 34, or second active compound 36, can be contained within its/their own set of microspheres and/or microcapsules. This is especially desirable where any one or more of compounds 32, 34 or 36 are liquids at typical atmospheric conditions.

Water 16, contained in microcapsules 14, is designed to initiate the reaction between the oxygen-releasing compound 32 and catalyst compound 34 upon the destruction of the shells of microcapsules 14. Additionally, the release of water 16 from microcapsules 14 wets second active compound 36, thereby enabling second active compound 36 to migrate towards the exterior of layer 30. Alternatively, microcapsules 14 could be eliminated where a water supply or other activating liquid exists externally of layer 30. This is especially true if material 12 is liquid-permeable, or even water-permeable.

In operation, layer 30 of FIG. 3 produces oxygen once microcapsules 14 are broken and/or decomposed via any suitable means (e.g., pressure, decomposition of the shells of the microcapsules by a suitable liquid, etc.), and the water contained therein is released thereby initiating a reaction between the oxygen releasing compound 32 and catalyst compound 34. The catalyzed reaction yields oxygen gas in an area within and/or surrounding layer 30. The oxygen gas produced within layer 30 escapes to the exterior of gas-permeable material 12. Additionally, the water released from microcapsules 14 also wets second active compound 36 and facilitates the migration of second active compound 36 to the exterior of layer 30. As would be apparent to one of skill in the art, layer 30 can be incorporated in a device designed to direct the flow of oxygen gas produced in layer 30 to a specific local or in a specific direction. Exemplary devices will be discussed in more detail below.

Referring specifically to FIG. 4, layer 40 of FIG. 4 differs from layer 30 of FIG. 3 in that layer 40 does not incorporate a second or additional active compounds. The embodiment of FIG. 4 is specifically engineered to deliver one liquid, gas and/or vapor phase compound rather than two or more liquid, gas and/or vapor phase compounds. The embodiment of FIG. 4 is advantageous in that it is specifically tailored to deliver one liquid, gas and/or vapor phase compound in a more cost effective manner when compared to the embodiment of FIG. 3.

In operation, layer 40 of FIG. 4 produces oxygen in a manner identical to that of layer 30 of FIG. 3. Accordingly, a detailed explanation of the workings of layer 40 is hereby omitted.

Referring specifically to FIG. 5, layer 50 of FIG. 5 differs from layer 30 of FIG. 3 in that layer 50 has microspheres and/or microcapsules 14a that contain catalyst compound 34. As with the embodiment of FIG. 3, the embodiment of FIG. 5 comprises one or more second active compounds 36, in solid or powder form, that are embedded within material 12. Alternatively, second active compound 36 can either be a solid or powder that is matrix encapsulated in microspheres, or a liquid that is microencapsulated in microcapsules.

In operation, layer 50 of FIG. 5 produces oxygen in a manner identical to that of layer 30 of FIG. 3. With regard to release of second active compound 36, this is accomplished by the break down or degradation of microspheres/microcapsules 14a by water 16. Alternatively, microspheres/microcapsules 14a can be designed to be broken down, degraded or decomposed by some liquid compound other than water or by some other physical, mechanical, or chemical means. Accordingly, a further detailed explanation of the workings of layer 50 is hereby omitted.

Referring specifically to FIG. 6, layer 60 of FIG. 6 differs from layer 50 of FIG. 5 in that layer 60 does not incorporate a second or additional active compounds. The embodiment of FIG. 6 is specifically engineered to deliver one liquid, gas and/or vapor phase compound rather than two or more liquid, gas and/or vapor phase compounds. The embodiment of FIG. 6 is advantageous in that it is specifically tailored to deliver one liquid, gas and/or vapor phase compound in a more cost effective manner when compared to the embodiment of FIG. 5.

In operation, layer 60 of FIG. 6 produces oxygen in a manner identical to that of layer 50 of FIG. 5. Accordingly, a detailed explanation of the workings of layer 60 is hereby omitted.

Referring specifically to FIG. 7, layer 70 of FIG. 7 differs from layer 50 of FIG. 5 in that layer 70 has microspheres and/or microcapsules 14b that contain a powder or solid oxygen releasing compound 32. As with the embodiment of FIG. 5, the embodiment of FIG. 7 comprises one or more second active compounds 36, in solid or powder form, that are embedded within material 12. Alternatively, second active compound 36 can either be a solid or powder that is matrix encapsulated in microspheres, or a liquid that is microencapsulated in microcapsules.

In operation, layer 70 of FIG. 7 produces oxygen in a manner identical to that of layer 50 of FIG. 5. With regard to release of second active compound 36, this is accomplished by the wetting of second active compound 36 by water 16 from microcapsules 14. Alternatively, second active compound 36 could be wetted by some liquid compound other than water. Accordingly, a further detailed explanation of the workings of layer 70 is hereby omitted.

Referring specifically to FIG. 8, layer 80 of FIG. 8 differs from layer 70 of FIG. 7 in that layer 80 does not incorporate a second or additional active compounds. The embodiment of FIG. 8 is specifically engineered to deliver one liquid, gas and/or vapor phase compound rather than two or more liquid, gas and/or vapor phase compounds. The embodiment of FIG. 8 is advantageous in that it is specifically tailored to deliver one liquid, gas and/or vapor phase compound in a more cost effective manner when compared to the embodiment of FIG. 7.

In operation, layer 80 of FIG. 8 produces oxygen in a manner identical to that of layer 70 of FIG. 7. Accordingly, a detailed explanation of the workings of layer 80 is hereby omitted.

Referring specifically to FIG. 9, layer 90 of FIG. 9 comprises microcapsules 14c that are incorporated in a gas-permeable material 12. Microcapsules 14c contain a liquid oxygen releasing compound 32 (e.g., hydrogen peroxide). In additional, material 12 contains or has embedded therein, in solid or powder form, a catalyst compound 34 (e.g., sodium permanganate, potassium permanganate, manganese (IV) oxide, etc.) and a second active compound 36. The second active compound 36 can be, for example, a blood thinning compound, an anti-coagulant, a pain relieving compound, a blood-clotting compound, an anti-bacterial compound, an anti-viral compound, or a pharmaceutical compound. As would be apparent to one of skill in the art, the embodiment of FIG. 9 is not limited to one second active compound. Rather, any one or more compounds that can be used as compound 16 can be used as additional active compounds, and can be embedded or deposited in material 12.

Alternatively, any one or all of catalyst compound 34 or second active compound 36 could be contained within its/their own set of microspheres and/or microcapsules. This is especially desirable where any one or more of compounds 34 or 36 are liquids at typical atmospheric conditions.

In operation, layer 90 of FIG. 9 produces oxygen once microcapsules 14 are broken and/or decomposed via any suitable means (e.g., pressure, decomposition of the shells of the microsphere by a suitable liquid, etc.), and the oxygen releasing compound 32 contained therein is released, thereby initiating a reaction between oxygen releasing compound 32 and catalyst compound 34. The catalyzed reaction yields oxygen gas in an area within and/or surrounding layer 90. The oxygen gas produced within layer 90 escapes to the exterior of gas-permeable material 12. Additionally, where oxygen releasing compound 32 is hydrogen peroxide, water is produced as a by-product of the catalyzed chemical reaction that yields the desired oxygen gas. The water by-product serves to wet second active compound 36 and facilitates compound 36's migration to the exterior of layer 90.

Referring specifically to FIG. 10, layer 100 of FIG. 10 differs from layer 90 of FIG. 9 in that layer 100 does not incorporate a second or additional active compounds. The embodiment of FIG. 10 is specifically engineered to deliver one liquid, gas and/or vapor phase compound rather than two or more liquid, gas and/or vapor phase compounds. The embodiment of FIG. 10 is advantageous in that it is specifically tailored to deliver one liquid, gas and/or vapor phase compound in a more cost effective manner when compared to the embodiment of FIG. 9.

In operation, layer 100 of FIG. 10 produces oxygen in a manner identical to that of layer 90 of FIG. 9. Accordingly, a detailed explanation of the workings of layer 100 is hereby omitted.

Referring specifically to FIG. 11, layer 110 of FIG. 11 comprises microspheres and/or microcapsules 14a and 14b that are incorporated in a gas-permeable material 12. Microspheres/microcapsules 14a contain catalyst compound 34, while microspheres/microcapsules 14b contain oxygen releasing compound 32. In the embodiment of FIG. 11 compounds 32 and 34 are in solid or powder form. However, the present invention is not limited thereto.

In additional, material 12 contains, or has embedded therein, in solid or powder form, a second active compound 36. The second active compound 36 can be, for example, a blood thinning compound, an anti-coagulant, a pain relieving compound, a blood-clotting compound, an anti-bacterial compound, an anti-viral compound, or a pharmaceutical compound. As would be apparent to one of skill in the art, the embodiment of FIG. 11 is not limited to one second active compound. Rather, any one or more compounds that can be used as compound 16 can be used as additional active compounds, and can be embedded or deposited in material 12.

In operation, layer 110 of FIG. 11 produces oxygen once microspheres and/or microcapsules 14a and 14b are broken and/or decomposed via any suitable means (e.g., pressure, decomposition of the shells of the microsphere by a suitable liquid, etc.). Upon the release of oxygen releasing compound 32 and catalyst compound 34, a reaction takes place that yields oxygen gas in an area within and/or surrounding layer 110. The oxygen gas produced within layer 110 escapes to the exterior of gas-permeable material 12. Additionally, where oxygen releasing composition 32 is hydrogen peroxide, water is produced as a by-product of the catalyzed chemical reaction that yields the desired oxygen gas. The water by-product serves to wet second active compound 36 and facilitates the migration of second active compound 36 to the exterior of layer 110.

Referring specifically to FIG. 12, layer 120 of FIG. 12 differs from layer 110 of FIG. 11 in that layer 120 does not incorporate a second or additional active compounds. The embodiment of FIG. 12 is specifically engineered to deliver one liquid, gas and/or vapor phase compound rather than two or more liquid, gas and/or vapor phase compounds. The embodiment of FIG. 12 is advantageous in that it is specifically tailored to deliver one liquid, gas and/or vapor phase compound in a more cost effective manner when compared to the embodiment of FIG. 11.

In operation, layer 120 of FIG. 12 produces oxygen in a manner identical to that of layer 110 of FIG. 11. Accordingly, a detailed explanation of the workings of layer 120 is hereby omitted.

Referring specifically to FIG. 13, layer 130 of FIG. 13 contains and/or has embedded therein, in solid or powder form, an oxygen-releasing compound 32, a catalyst compound 34 and a second active compound 36 in a material 12. Compounds 32, 34 and 36 can be selected from the same compounds discussed above in relation the embodiment of FIG. 3.

Material 12 is both gas and liquid-permeable (particularly water-permeable). The second active compound 36 can be, for example, a blood thinning compound, an anti-coagulant, a pain relieving compound, a blood-clotting compound, an anti-bacterial compound, an anti-viral compound, or a pharmaceutical compound. As would be apparent to one of skill in the art, the embodiment of FIG. 13 is not limited to one second active compound. Rather, any one or more compounds that can be used as compound 16 can be used as additional active compounds, and can be embedded or deposited in material 12.

Alternatively, any one or all of oxygen-releasing compound 32, catalyst compound 34, or second active compound 36 could be contained within in their own set of microspheres and/or microcapsules. This is especially desirable where any one or more of compounds 32, 34 or 36 are liquids at typical atmospheric conditions.

In operation, layer 130 of FIG. 13 produces oxygen once material 12 is subjected to a liquid (e.g., water, blood, saliva, urine, etc.). The liquid permeates into material 12 thereby initiating a reaction between oxygen releasing compound 32 and catalyst compound 34. In addition, the liquid activates second active compound 36 and facilitates delivery of compound 36 to the exterior of layer 130. Alternatively, if any of all of compounds 32, 34 and 36 are contained within microspheres and/or microcapsules, the liquid breaks down the shells of such microcapsules, or the microspheres themselves, thereby initiating the above-mentioned reaction between oxygen releasing compound 32 and catalyst 34.

Referring specifically to FIG. 14, layer 140 of FIG. 14 differs from layer 130 of FIG. 13 in that layer 140 does not incorporate a second or additional active compounds. The embodiment of FIG. 14 is specifically engineered to deliver one liquid, gas and/or vapor phase compound rather than two or more liquid, gas and/or vapor phase compounds. The embodiment of FIG. 14 is advantageous in that it is specifically tailored to deliver one liquid, gas and/or vapor phase compound in a more cost effective manner when compared to the embodiment of FIG. 13.

In operation, layer 140 of FIG. 14 produces oxygen in a manner identical to that of layer 130 of FIG. 13. Accordingly, a detailed explanation of the workings of layer 140 is hereby omitted.

Exemplary Films Incorporating Liquid, Gas and/or Vapor Phase Delivery Layers

Referring now to FIGS. 15A through 18, FIGS. 15A through 18 disclose different embodiments of a film which incorporates therein an oxygen gas producing layer according to the present invention. FIG. 15A and FIGS. 16 through 18 illustrate films that include therein gas, liquid and/or vapor phase compound producing layer 30 of FIG. 3. FIG. 15B illustrates a film that includes therein an alternative gas, liquid and/or vapor phase producing layer 30a. However, as would be apparent to one of skill in the art, the embodiments of FIG. 15A and FIGS. 16 through 18 are not limited to the inclusion of only layer 30. Rather, the embodiments of FIG. 15A through 18 can include at least one of any of the liquid, gas and/or vapor phase compound producing layers disclosed herein. In addition, the embodiments of FIGS. 15A through 18 are not limited to solely the inclusion of layers that generate oxygen gas, or oxygen gas in addition to at least one other compound. As noted above, the liquid, gas and/or vapor phase producing layers of the present invention can be designed to produce a wide variety of compounds or combination of compounds.

Referring specifically to FIG. 15A, FIG. 15A illustrates a liquid, gas and/or vapor phase compound producing film according to one embodiment of the present invention. From top to bottom as illustrated in FIG. 15A, film 150 comprises a gas-impermeable and liquid-impermeable layer 152, liquid, gas and/or vapor phase compound producing layer 30, a water-impermeable/gas-permeable layer 154, and non-stick layer 156. As noted above, film 150 is not limited to just the production of oxygen, or the combinations of oxygen and additional compounds discussed above. Rather, film 150 can be designed to produce any desired liquid, gas and/or vapor phase compound disclosed herein.

Although film 150 is illustrated with one liquid, gas and/or vapor phase compound producing layer, films comprising two or more liquid, gas and/or vapor phase compound producing layers are also within the scope of the present invention. In the case where two or more liquid, gas and/or vapor phase compound producing layers are present in a film, each layer can produce one or more liquid, gas and/or vapor phase compounds. As would be obvious to one of skill in the art, where two or more liquid, gas and/or vapor phase compound producing layers are present, the layers can produce the same or different single or multiple liquid, gas and/or vapor phase compounds.

Any suitable compound can be used to form gas-impermeable and liquid-impermeable layer 152 so long as the compound used is both impermeable to one or more gases and one or more liquids. Compounds that can be used to form gas-impermeable and liquid-impermeable layer 152 include, but not limited to, polymers, co-polymers, terpolymers, block polymers, and block co-polymers.

Suitable polymers for use as material 152 include, but are not limited to, polyolefins, polyethylenes, polystyrenes, polypropylenes, polyurethanes, polymethacrylates, degradable polymers, biodegradable polymers, starch-based polymers, polyvinyl alcohols, polyvinyl acetates, polyenlketones, or co-polymer combinations of two or more thereof.

Biodegradable suitable for layer 152 include, but are not limited to, polyhydroxy-alkanoates (PHA), such as polyhydroxybutyrate (PHB), linear ε-polycaprolactone (PCL), or copolymers of polyhydroxybutyrate and polyhydroxyvalerate (PHBV), polylatic acid polymers, polyglycolic acid polymers, biodegradable polyester amide polymers, biodegradable polyester urethane polymers and biodegradable copolymers of any combination of two or more of the above. Such copolymers could include two or more of the same type of polymer, for example, two or more different biodegradable polyesters.

Layer 30 is described in detail above, and therefore a detailed description thereof is omitted here for the sake of brevity.

Any suitable compound can be used to form water-impermeable/gas-permeable layer 154 so long as the compound used is permeable to one or more gases and impermeable to at least water. In another embodiment, water-impermeable/gas-permeable layer 154 is not only impermeable to water, but layer 154 is impermeable to one or more liquids in addition to water (e.g., blood, urine, puss, saliva, etc.). Compounds that can be used to form water-impermeable/gas-permeable layer 154 include, but not limited to, polymers, co-polymers, terpolymers, block polymers, block co-polymers, adhesives, and gels.

In one embodiment, water-impermeable/gas-permeable layer 154 is formed from a polymer, co-polymer, terpolymer, or block co-polymer layer. Suitable polymers for use as layer 154 include, but are not limited to, polyolefins, polyethylenes, polystyrenes, polypropylenes, polyurethanes, polymethacrylates, degradable polymers, biodegradable polymers, starch-based polymers, polyvinyl alcohols, polyvinyl acetates, polyenlketones, or co-polymer combinations of two or more thereof.

Biodegradable suitable for layer 154 include, but are not limited to, polyhydroxy-alkanoates (PHA), such as polyhydroxybutyrate (PHB), linear ε-polycaprolactone (PCL), or copolymers of polyhydroxybutyrate and polyhydroxyvalerate (PHBV), polylatic acid polymers, polyglycolic acid polymers, biodegradable polyester amide polymers, biodegradable polyester urethane polymers and biodegradable copolymers of any combination of two or more of the above. Such copolymers could include two or more of the same type of polymer, for example, two or more different biodegradable polyesters.

Any suitable compound can be used to non-stick layer 156 so long as the compound used does not stick to any surface and/or object that comes into contact with film 150, and the compound used for layer 156 is at least gas-permeable to permit the escape of the gas generated in layer 30 of film 150. In another embodiment, layer 156 is both gas and liquid-permeable. Compounds that can be used to form non-stick layer 156 include, but not limited to, polymers, co-polymers, terpolymers, block polymers, block co-polymers, open-celled foams, closed-cell foams, silicone containing compositions (e.g., silicon containing polymer compositions), and Teflon.

In one embodiment, non-stick layer 156 is formed from a polymer, co-polymer, terpolymer, or block co-polymer layer. Suitable polymers for use as layer 156 include, but are not limited to, polyolefins, polyethylenes, polystyrenes, polypropylenes, polyurethanes, polymethacrylates, degradable polymers, biodegradable polymers, starch-based polymers, polyvinyl alcohols, polyvinyl acetates, polyenlketones, or co-polymer combinations of two or more thereof.

Biodegradable suitable for layer 156 include, but are not limited to, polyhydroxy-alkanoates (PHA), such as polyhydroxybutyrate (PHB), linear ε-polycaprolactone (PCL), or copolymers of polyhydroxybutyrate and polyhydroxyvalerate (PHBV), polylatic acid polymers, polyglycolic acid polymers, biodegradable polyester amide polymers, biodegradable polyester urethane polymers and biodegradable copolymers of any combination of two or more of the above. Such copolymers could include two or more of the same type of polymer, for example, two or more different biodegradable polyesters.

In another embodiment, the compound used for layer 156 is an open or closed-cell foam. Suitable compositions that may be used to produce foams (either open-cell, closed-cell, or both) include, but are not limited to, acrylonitrile butadiene styrene (ABS), polyvinyl chlorides (PVCs), polyurethanes, polypropylenes, crosslinkable polymer compositions, polystyrenes, polyethylenes, polyolefins, and co-polymers of at least two polyolefins.

Film 150 can be formed from any suitable film forming technique. Suitable techniques include, but are not limited to extrusion, co-extrusion, and casting techniques. All of the layers contained in film 150 do not have to be produced simultaneously. Rather, sub-portions of film 150 can be produced and then joined together in a later process step. For example layers 152 and 154 could be formed together and, in a separate process step, layers 156 and 158 could be formed together. The two sub-portions are then joined together to yield film 150.

In operation, film 150 produces oxygen in a manner identical to layer 30, as explained above except that the microcapsules in layer 30 are broken and/or decomposed via pressure, via a gas and/or a liquid other than water (in the case where layer 154 is only water-impermeable). Once the microcapsules in layer 30 have released the water contained therein, oxygen gas is produced in layer 30 as is explained above. The difference with film 150 is that the oxygen gas produced in layer 30 is permitted to escape in substantially only one direction, through layers 154 and 156. Accordingly, if film 150 is formed into some type of enclosure with the gas producing side facing inward, film 150 can be used to produce an increased concentration of oxygen within the interior of the enclosure formed by film 150. In another embodiment, a concentration and/or an increased concentration of a liquid, gas and/or vapor phase compound or compounds, other than or in addition to oxygen, can be produced in the interior of an enclosure formed by film 150 so long as the film is designed to permit the unidirectional escape of the desired liquid, gas and/or vapor phase composition or compositions.

Referring specifically to FIG. 15B, FIG. 15B illustrates an alternative embodiment 150a of a liquid, gas and/or vapor phase compound producing film according to the present invention. The embodiment of FIG. 15B differs from the embodiment of FIG. 15A in that the film of FIG. 15B comprises a liquid, gas and/or vapor phase compound producing layer 30a formed from the combination of a layer of microspheres and/or microcapsules 157 and anchoring layers 158a and 158b. As can be seen from FIG. 15B, the microspheres and/or microcapsules contained in film 150a are partially anchored in both the upper and lower anchoring layers 158a and 158b, respectively. The amount of the microsphere and/or microcapsule contained within layer 158a and/or 158b is unimportant, so long as the microspheres and/or microcapsules of film 150a are anchored securely enough to main the structural integrity of film 150a.

The size-of the microspheres and/or microcapsules contained in layer 157 are chosen so as to create an air gap 159 between upper anchoring layer 158a and lower anchoring layer 158b. Anchoring layers 158a and 158b can be formed from any suitable polymer, co-polymer, terpolymer, block polymer, block co-polymer, adhesive, hot-melt adhesive or gel. In one embodiment layers 158a and 158b are selected from any suitable adhesive, hot-melt adhesive, or thermoplastic polymer in order to form anchoring layers 158a and 158b to anchor the microspheres and/or microcapsules contained within film 150a therein. The remaining layers of film 150a are identical to their respective layers in film 150. Accordingly, a detailed discussion thereof is omitted.

Film 150a can be formed from any suitable film forming technique. Suitable techniques include, but are not limited to extrusion, co-extrusion, and casting techniques. Given the presence of air gap 159 in film 150a, film 150a lends itself to being created in two separate sub-portions and then assembled after the microspheres and/or microcapsules contained in film 150a are deposited, via a suitable technique, in either one or both of anchoring layers 158a and 158b. Ideally, the microspheres and/or microcapsules contained in film 150a are deposited on either one or the other of anchoring layers 158a and 158b, thereby eliminating the possibility that some or all of the microspheres and/or microcapsules contained in film 150a are only anchored in one of layers 158a or 158b, rather than being anchored in both layer 158a and 158b.

In operation, film 150a produces oxygen in a manner identical to that of film 150 of FIG. 15A. Therefore, a detailed discussion of the operation of film 150a is hereby omitted for the sake of brevity.

As would be apparent to one of skill in the art, layer 30a can be used in any of the embodiments where layer 30 is used.

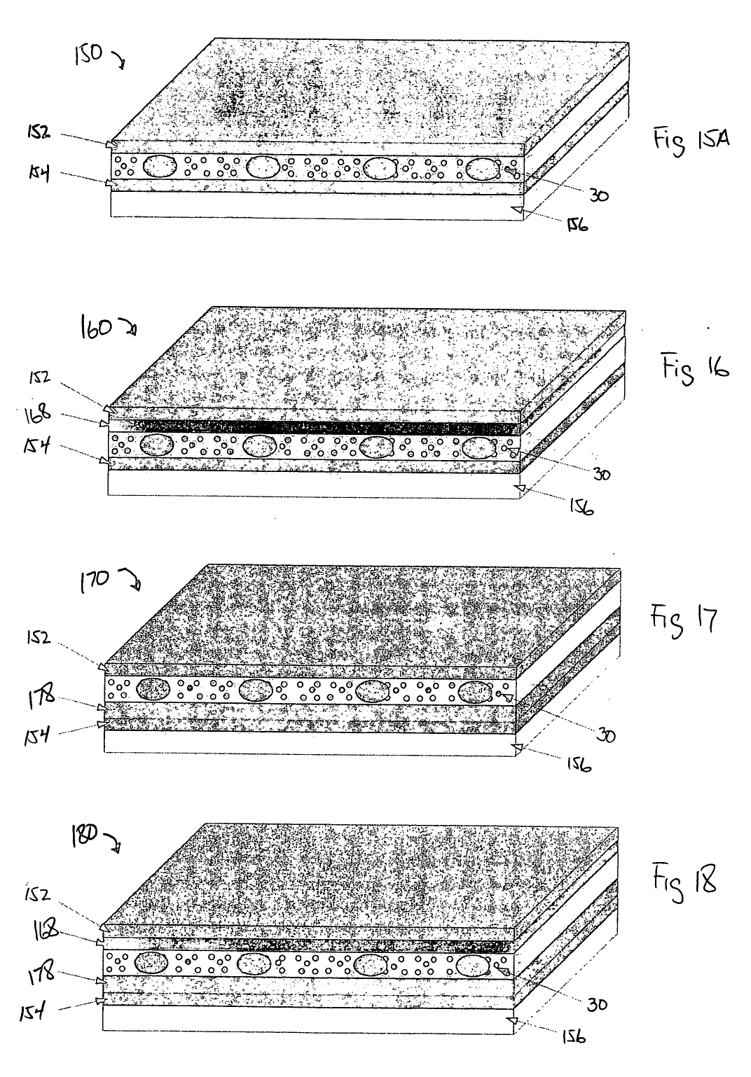

Referring specifically to FIG. 16, FIG. 16 illustrates a liquid, gas and/or vapor phase compound producing film 160 according to another embodiment of the present invention. Film 160 of FIG. 16 differs from film 150 of FIG. 15 in that film 160 includes therein a layer 168 of sand paper between gas-impermeable and liquid-impermeable layer 152 and liquid, gas and/or vapor phase compound producing layer 30. The sand paper layer 168 helps to break the microcapsules contained in layer 30, thereby releasing the water contained therein. Sand paper layer 168 breaks the microcapsules contained in layer 30 when film 160 undergoes, for example, bending, twisting, or pressure.

In operation, film 160 produces oxygen in a manner identical to that of layer 30 in film 150, except the need for a gas and/or liquid other than water to be present to break and/or decompose the microcapsules in layer 30 is substantially reduced and/or eliminated.

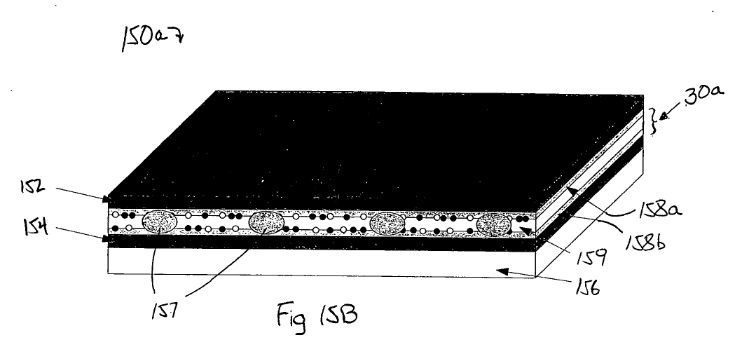

Referring specifically to FIG. 17, FIG. 17 illustrates a liquid, gas and/or vapor phase compound producing film 170 according to another embodiment of the present invention. Film 170 of FIG. 17 differs from film 150 of FIG. 15 in that film 170 includes therein a layer 178 of material 12 that has been impregnated and/or contains at least one second active compound (e.g., a pain-killing or relieving compound) positioned between layer 30 and layer 154. As is discussed above, any one or more compounds that can be used as compound 16 can be used as one or more second active compounds, and can be embedded or deposited in material 12 of layer 178. Material 12 as chosen to be used in layer 178 should be, at a minimum, both water and gas-permeable. In another embodiment, material 12 as chosen to be used in layer 178 can be both liquid and gas-permeable.

In operation, film 170 produces oxygen in a manner identical to that of layer 30 in film 150, except the water produced in layer 30 also facilitates the delivery of the second active compound through layers 154 and 156 to the bottom exterior surface of film 170.

Referring specifically to FIG. 18, FIG. 18 illustrates a liquid, gas and/or vapor phase compound producing film 180 according to another embodiment of the present invention. Film 180 of FIG. 18 differs from film 160 of FIG. 16 in that film 180 includes therein a sand paper layer 168 position between layer 152 and layer 30, and a layer 178 of material 12 that has been impregnated and/or contains at least one second active compound (e.g., a pain-killing or relieving compound) positioned between layer 30 and layer 154. As is discussed above, any one or more compounds that can be used as compound 16 can be used as one or more second active compounds, and can be embedded or deposited in material 12 of layer 178. Material 12 as chosen to be used in layer 178 should be, at a minimum, both water and gas-permeable. In another embodiment, material 12 as chosen to be used in layer 178 can be both liquid and gas-permeable.

In operation, film 180 produces oxygen in a manner identical to that of layer 30 in film 160, except the water produced in layer 30 also facilitates the delivery of the second active compound through layers 154 and 156 to the bottom exterior surface of film 180.

Exemplary Uses for the Liquid, Gas and/or Vapor Phase Producing Layers and/or Films

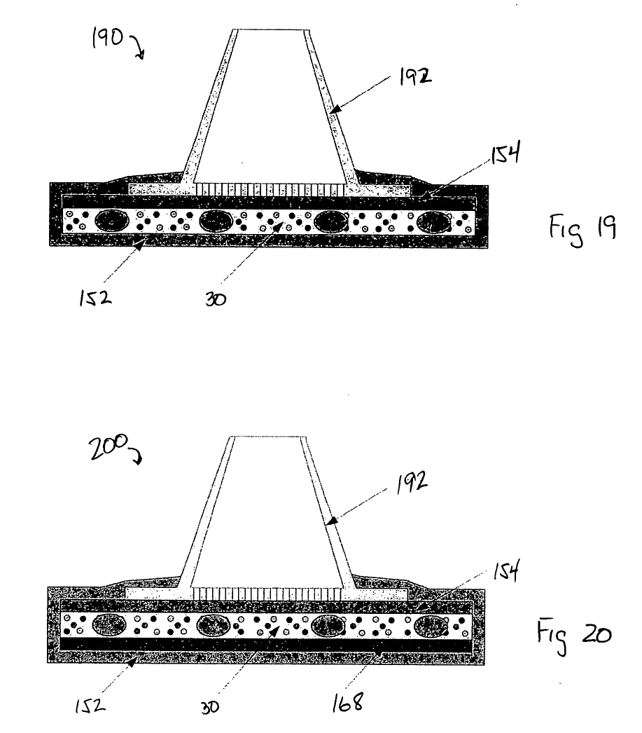

The liquid, gas and/or vapor phase compound producing films of the present invention can be incorporated into any structure, device, and/or packaging where it is desired to produce a concentration of, or increase the concentration of, at least one liquid, gas and/or vapor phase composition. Some examples include, but are not limited to, packaging for food, films for lining planters, pots or other horticulture structures, soil spikes for use in horticulture or farming, packaging for cosmetics, pharmaceuticals, dietary supplements beverages, or food stuffs, bags or enclosure for containing waste, bio-hazardous waste, gas masks, or liquid, gas and/or vapor phase compound producing film-based capsules (see FIGS. 19 and 20 and the related text below).

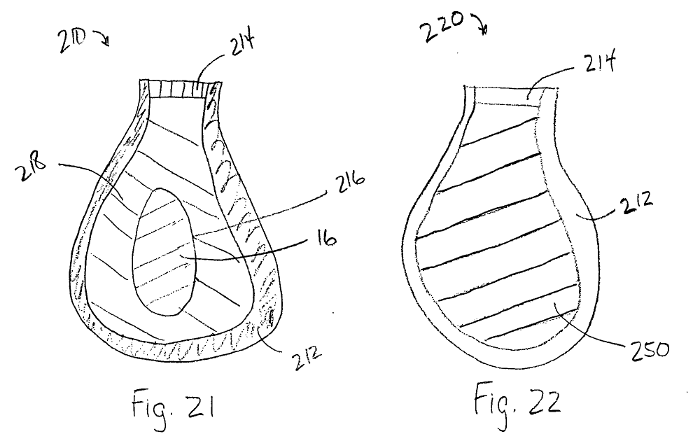

The present invention can be used to produce a concentration of and/or increase the concentration of any desired liquid, gas and/or vapor phase composition in a local area or enclosure. For example, an oxygen producing capsule according to the present invention could be used to increase the oxygen concentration in a room, a container or a liquid (e.g., paint, water, gels, cosmetics, lotions, creams, oil, diesel fuel, etc.). Examples of oxygen producing film-based capsules are shown in FIGS. 19 and 20. Examples of non-film based capsules are shown in FIGS. 21 and 22.

In one embodiment, the liquid, gas and/or vapor phase compound producing systems and/or films of the present invention permit the in-situ (i.e., self-contained) production of at least one liquid, gas and/or vapor phase compound in a liquid, gas and/or vapor phase compound producing layer (e.g., layers 30, 40, 50, 60, 70, 80, 90, 100, 110, 120, 130 and 140).

Film-Based Capsules

Referring specifically to FIG. 19, FIG. 19 illustrates an oxygen producing film-based capsule 190 formed from film 150 of FIG. 15. In capsule 190, non-stick layer 156 has been eliminated and replaced with a pipe structure, a one-way valve or straw 192. Although not limited thereto, FIG. 19 depicts structure 192 as a pipe. Additionally, layer 152 has been formed to completely surround capsule 190 except for the opening provided by pipe 192. Layer 30 of capsule 190 produces oxygen in a manner identical to that of film 150. Once produced, the oxygen formed in layer 30 is funneled out of capsule 190 by pipe 192.

Referring specifically to FIG. 20, FIG. 20 illustrates an oxygen producing film-based capsule 200 formed from film 160 of FIG. 16. In capsule 200, non-stick layer 156 has been eliminated and replaced with a pipe structure 192. Additionally, layer 152 has been formed to completely surround capsule 200 except for the opening provided by pipe 192. Layer 30 of capsule 200 produces oxygen in a manner identical to that of film 160. Once produced, the oxygen formed in layer 30 is funneled out of capsule 200 by pipe 192.

Alternatively, pipe 192 of FIGS. 19 and 20 can be eliminated and one or more openings (not shown) can be left in layer 152 to permit the escape of the one or more liquid, -gas and/or vapor phase compositions produced in layer 30. In another embodiment, the one or more openings in layer 152 can be covered by a removable seal (not shown). Such seals are known in the art and a discussion hereof is omitted for brevity.

Non-Film Based Capsules

In another embodiment of the present invention, gas, liquid, and/or vapor phase producing layer 30 or 30a can be replaced by one or more gas, liquid, and/or vapor phase producing capsules (see FIGS. 21 and 22). In the case where layer 30 and/or 30a is present in a film or other structure, the gas, liquid, and/or vapor phase producing capsule of the present invention can be designed to include a suitable delivery means (e.g., via a pipe, tube, or micro-capillary pipette, or a syringe) in order to deliver the desired gas, liquid and/or vapor phase compound to the appropriate portion or portions of the film or other structure.

Referring specifically to FIG. 21, FIG. 21 is a cross-section illustration of a gas, liquid, and/or vapor phase compound producing capsule 210. Capsule 210 comprises a gas-impermeable and liquid-impermeable shell 212, a one-way vent 214, an interior storage capsule 216, and a reaction layer 218.

Shell 212 can be formed from any suitable compound so long as the compound is both gas-impermeable and liquid-impermeable. Compounds that can be used to form gas-impermeable and liquid-impermeable shell 212 include, but not limited to, polymers, co-polymers, terpolymers, block polymers, block co-polymers, or rubber.

Suitable polymers for use in forming shell 212 include, but are not limited to, polyolefins, polyethylenes, polystyrenes, polypropylenes, polyurethanes, polymethacrylates, degradable polymers, biodegradable polymers, starch-based polymers, polyvinyl alcohols, polyvinyl acetates, polyenlketones, or co-polymer combinations of two or more thereof.

Biodegradable suitable for use in forming shell 212 include, but are not limited to, polyhydroxy-alkanoates (PHA), such as polyhydroxybutyrate (PHB), linear ε-polycaprolactone (PCL), or copolymers of polyhydroxybutyrate and polyhydroxyvalerate (PHBV), polylatic acid polymers, polyglycolic acid polymers, biodegradable polyester amide polymers, biodegradable polyester urethane polymers and biodegradable copolymers of any combination of two or more of the above. Such copolymers could include two or more of the same type of polymer, for example, two or more different biodegradable polyesters.

Interior storage capsule 216 is a breakable capsule formed from any suitable compound (e.g., glass, a polymer, etc.) and contains therein any one or more of compounds 16, as described above. As discussed with regard to FIGS. 21 and 22, compound 16 is an oxygen gas producing compound (e.g., hydrogen peroxide). However, the embodiment of FIGS. 21 and 22 are not limited thereto. Rather, the capsules of FIGS. 21 and 22 can be designed to produce a wide variety of gas, liquid and/or vapor phase compounds. In one embodiment, interior storage capsule 216 is formed so as to be breakable by hand.

Reaction layer 218 comprises at least one catalyst (e.g., potassium permanganate) to facilitate the production of the desired gas, liquid, and/or vapor phase compound. Reaction layer 218 can further comprise at least one second active compound. The second active compound can be, for example, a blood thinning compound, an anti-coagulant, a pain relieving compound, a blood-clotting compound, an anti-bacterial compound, an anti-viral compound, or a pharmaceutical compound. As would be apparent to one of skill in the art, the embodiment of FIG. 21 is not limited to one second active compound. Rather, any one or more compounds that can be used as compound 16 can be used as additional active compounds.

In order to generate the desired gas, liquid, and/or vapor phase compound or compounds, a user applies pressure to capsule 210 to break interior storage capsule 216 and release oxygen gas producing hydrogen peroxide compound 16. The hydrogen peroxide reacts with the catalyst contained in reaction layer 218 to yield oxygen gas. The oxygen gas escapes via one way vent 214 to the exterior of capsule 210.

Capsule 210 can be designed to be used as a personal source of oxygen gas that can be, for example, inhaled orally. Alternatively, capsule 210 can be designed to replace and/or supplement the liquid, gas and/or vapor phase compound production of layers 30 and/or 30a of FIGS. 15A through 18. In the instance where capsule 210 is being used to replace layers 30 and/or 30a, layers 30 and/or 30a can themselves be replaced by a gas, liquid, and/or vapor phase compound dispensing means (e.g., a rubber bladder having a series of holes therein) that is connected via any suitable delivery means to capsule 210 in order to ensure that the desired liquid, gas and/or vapor phase compound is delivered to the desired location. Such an arrangement permits the gas, liquid, and/or vapor phase compound producing portion of the present invention to be located outside of the remainder of the delivery system of the present invention.

Referring specifically to FIG. 22, FIG. 22 is a cross-section illustration of a gas, liquid, and/or vapor phase compound producing capsule 220. Capsule 220 differs from capsule 210 of FIG. 21 in that interior storage capsule 216 and a reaction layer 218 have been replaced by combined core 250. Combined core 250 contains a combination of microspheres and/or microcapsules 14 with at least one compound selected from a catalyst or a second active agent. Microspheres/microcapsules 14 contain one or more compound 16, as described above. The operation of combined core 250 is identical to the operation of layer 30 where at least one compound is contained within a set of microspheres/microcapsules 14. The gas, liquid, and/or vapor phase compound produced within combined core 250 escapes from capsule 220 via one-way vent 214. As with capsule 210, capsule 220 can be used as a substitute or replacement for layers 30 and/or 30a of the embodiments of FIGS. 15A through 18.

While the present application illustrates various embodiments, and while these embodiments have been described in some detail, it is not the intention of the applicant to restrict or in any way limit the scope of the claimed invention to such detail. Additional advantages and modifications will readily appear to those skilled in the art. Therefore, the invention, in its broader aspects, is not limited to the specific details, the representative apparatus, and illustrative examples shown and described. Accordingly, departures may be made from such details without departing from the spirit or scope of the applicant's claimed invention.

1. A liquid, gas and/or vapor phase producing layer comprising:
at least one compound embedded in the liquid, gas and or vapor phase producing layer that is capable of producing a liquid, gas and/or vapor phase compound,
wherein the least one compound that produces the liquid, gas and/or vapor phase compound has a shelf-life of at least about 3 months as a result of being embedded in the liquid, gas and/or vapor phase producing layer.

2. The liquid, gas and/or vapor phase producing layer of claim 1, wherein the at least one compound that produces the liquid, gas and/or vapor phase compound is contained within microspheres, microcapsules, or a combination of microspheres and microcapsules, and the microspheres and/or microcapsules are embedded in the liquid, gas and/or vapor phase compound producing layer.

3. The liquid, gas and/or vapor phase producing layer of claim 2, wherein the microspheres are formed from a compound selected from waxes, hydrocolloids, polyethylene polymers, polypropylene polymers, polymethacrylates polymers, polyester polymers, polyurethane polymers, polyurethane/polyurea co-polymers, polyurea polymers, polyethersulfone polymers, thermoplastic polymers, one or more non-curing components of a thermosetting polymer, or combinations of two or more thereof.

4. The liquid, gas and/or vapor phase producing layer of claim 2, wherein the microspheres are formed from a compound selected from ZrO₂, HfO₂, SiO₂, Al₂O₃, ZrHfO₄, or combinations of two or more thereof.

5. The liquid, gas and/or vapor phase producing layer of claim 2, wherein the microcapsules are formed from a compound selected from waxes, hydrocolloids, polyethylene polymers, polypropylene polymers, polymethacrylates polymers, polyester polymers, polyurethane polymers, polyurethane/polyurea co-polymers, polyurea polymers, polyethersulfone polymers, thermoplastic polymers, one or more non-curing components of a thermosetting polymer, or combinations of two or more thereof.

6. The liquid, gas and/or vapor phase producing layer of claim 2, wherein the microcapsules are formed from a compound selected from ZrO₂, HfO₂, SiO₂, Al₂O₃, ZrHfO₄, or combinations of two or more thereof.

7. The liquid, gas and/or vapor phase producing layer of claim 1, wherein the liquid, gas and/or vapor phase producing layer is formed from a compound selected from polymers, co-polymers, terpolymers, block polymers, open-celled foams, closed-cell foams, paper, cellulose, adhesives, or gels.

8. The liquid, gas and/or vapor phase producing layer of claim 7, wherein the liquid, gas and/or vapor phase producing layer is formed from one or more polyolefin polymers, polyethylene polymers, polystyrene polymers, polypropylene polymers, polyurethane polymers, polymethacrylates polymers, degradable polymers, biodegradable polymers, starch-based polymers, polyvinyl alcohols, polyvinyl acetates, polyenlketones, or co-polymer combinations of two or more thereof.

9. The liquid, gas and/or vapor phase producing layer of claim 8, wherein the liquid, gas and/or vapor phase producing layer is formed from one or more biodegradable polymers selected from polyhydroxy-alkanoates (PHA), polyhydroxybutyrate (PHB), linear ε-polycaprolactone (PCL), or copolymers of polyhydroxybutyrate and polyhydroxyvalerate (PHBV), polylatic acid polymers, polyglycolic acid polymers, biodegradable polyester amide polymers, biodegradable polyester urethane polymers and biodegradable copolymers of any combination of two or more of the above.

10. The liquid, gas and/or vapor phase producing layer of claim 7, wherein the liquid, gas and/or vapor phase producing layer is an open or closed-cell foam layer.

11. The liquid, gas and/or vapor phase producing layer of claim 10, wherein the liquid, gas and/or vapor phase producing layer is formed from at least one compound selected from acrylonitrile butadiene styrene (ABS), polyvinyl chlorides (PVCs), polyurethanes, polypropylenes, crosslinkable polymer compositions, polystyrenes, polyethylenes, polyolefins, and co-polymers of at least two polyolefins.

12. The liquid, gas and/or vapor phase producing layer of claim 1, wherein the compound that produces the liquid, gas and/or vapor phase compound is selected from one or more oxygen gas-producing compounds, nitrogen gas-producing compounds, vapor phase corrosion inhibiting compounds, water, anti-bacterial compounds, anti-viral compounds, anti-static compounds, disinfectants, pain-reliving compounds, anti-coagulant compounds, blood-thinning compounds, blood clotting compounds/promoters, fragrance compounds, stimulants, vitamins, amino-acid supplements, skin-care products, compounds designed to treat acne, odor suppressants, odor enhancing compounds, pharmaceutical compounds, UV-protectant compounds, lubricant compounds, fertilizers, polishing compounds, cleaning compounds, flavor compounds, citrus extracts, medicinal compounds, or compatible mixtures of two or more thereof.

13. The liquid, gas and/or vapor phase producing layer of claim 12, wherein the compound that produces the liquid, gas and/or vapor phase compound is selected from lithium perchlorate, sodium perchlorate, potassium perchlorate, lithium peroxide, sodium peroxide, potassium peroxide, calcium peroxide, magnesium peroxide, barium peroxide, lead peroxide, carbamide peroxide (CH₆N₂O₃), potassium nitrate, potassium permanganate, chromium (VI) oxide, potassium dichromate, and mixtures of two or more thereof.

14. The liquid, gas and/or vapor phase producing layer of claim 12, wherein the liquid, gas and/or vapor phase producing layer further comprises at least one catalyst that facilitates the production of the liquid, gas and/or vapor phase compound from the liquid, gas and/or vapor phase producing layer.

15. The liquid, gas and/or vapor phase producing layer of claim 14, wherein the at least one catalyst is selected from sodium permanganate, potassium permanganate, manganese (IV) oxide, or mixtures of two or more thereof.

16. The liquid, gas and/or vapor phase producing layer of claim 1, wherein the layer is contained within a capsule, bandage, packing, or film that produces at least one liquid, gas and/or vapor phase compound.

17. A liquid, gas and/or vapor phase producing film comprising:
a gas-impermeable and liquid-impermeable layer having a first surface and a second surface;
a liquid, gas and/or vapor phase producing layer having a first surface and a second surface, the first surface of the liquid, gas and/or vapor phase producing layer facing the second surface of the gas-impermeable and liquid-impermeable layer; and
a water-impermeable, gas-permeable layer having a first surface and a second surface, the first surface of the water-impermeable, gas-permeable layer facing the second surface of the liquid, gas and/or vapor phase producing layer,
wherein the liquid, gas and/or vapor phase producing layer has embedded therein at least one compound that is capable of producing a liquid, gas and/or vapor phase compound, and the least one compound that produces the liquid, gas and/or vapor phase compound has a shelf-life of at least about 3 months as a result of being embedded in the liquid, gas and/or vapor phase producing layer.

18. The liquid, gas and/or vapor phase producing film of claim 17, wherein the gas-impermeable and liquid-impermeable is formed form a compound selected from polymers, co-polymers, terpolymers, block polymers, and block co-polymers.

19. The liquid, gas and/or vapor phase producing film of claim 17, wherein the gas-impermeable and liquid-impermeable is formed form a compound selected from polyolefin polymers, polyethylene polymers, polystyrene polymers, polypropylene polymers, polyurethane polymers, polymethacrylates polymers, degradable polymers, biodegradable polymers, starch-based polymers, polyvinyl alcohols, polyvinyl acetates, polyenlketones, or co-polymer combinations of two or more thereof.

20. The liquid, gas and/or vapor phase producing film of claim 17, wherein the at least one compound that produces the liquid, gas and/or vapor phase compound is contained within microspheres, microcapsules, or a combination of microspheres and microcapsules, and the microspheres and/or microcapsules are embedded in the liquid, gas and/or vapor phase compound producing layer.

21. The liquid, gas and/or vapor phase producing film of claim 20, wherein the microspheres are formed from a compound selected from waxes, hydrocolloids, polyethylene polymers, polypropylene polymers, polymethacrylates polymers, polyester polymers, polyurethane polymers, polyurethane/polyurea co-polymers, polyurea polymers, polyethersulfone polymers, thermoplastic polymers, one or more non-curing components of a thermosetting polymer, or combinations of two or more thereof.

22. The liquid, gas and/or vapor phase producing film of claim 20, wherein the microspheres are formed from a compound selected from ZrO₂, HfO₂, SiO₂, Al₂O₃, ZrHfO₄, or combinations of two or more thereof.

23. The liquid, gas and/or vapor phase producing film of claim 20, wherein the microcapsules are formed from a compound selected from waxes, hydrocolloids, polyethylene polymers, polypropylene polymers, polymethacrylates polymers, polyester polymers, polyurethane polymers, polyurethane/polyurea co-polymers, polyurea polymers, polyethersulfone polymers, thermoplastic polymers, one or more non-curing components of a thermosetting polymer, or combinations of two or more thereof.

24. The liquid, gas and/or vapor phase producing film of claim 20, wherein the microcapsules are formed from a compound selected from ZrO₂, HfO₂, SiO₂, Al₂O₃, ZrHfO₄, or combinations of two or more thereof.

25. The liquid, gas and/or vapor phase producing film of claim 17, wherein the liquid, gas and/or vapor phase producing layer is formed from a compound selected from polymers, co-polymers, terpolymers, block polymers, open-celled foams, closed-cell foams, paper, cellulose, adhesives, or gels.

26. The liquid, gas and/or vapor phase producing film of claim 25, wherein the liquid, gas and/or vapor phase producing layer is formed from a compound selected from polyolefin polymers, polyethylene polymers, polystyrene polymers, polypropylene polymers, polyurethane polymers, polymethacrylates polymers, degradable polymers, biodegradable polymers, starch-based polymers, polyvinyl alcohols, polyvinyl acetates, polyenlketones, or co-polymer combinations of two or more thereof.

27. The liquid, gas and/or vapor phase producing film of claim 26, wherein the liquid, gas and/or vapor phase producing layer is formed from a compound selected from polyhydroxy-alkanoates (PHA), polyhydroxybutyrate (PHB), linear ε-polycaprolactone (PCL), or copolymers of polyhydroxybutyrate and polyhydroxyvalerate (PHBV), polylatic acid polymers, polyglycolic acid polymers, biodegradable polyester amide polymers, biodegradable polyester urethane polymers and biodegradable copolymers of any combination of two or more of the above.

28. The liquid, gas and/or vapor phase producing film of claim 25, wherein the liquid, gas and/or vapor phase producing layer is an open or closed-cell foam layer.

29. The liquid, gas and/or vapor phase producing film of claim 28, wherein the liquid, gas and/or vapor phase producing layer is formed at least one compound selected acrylonitrile butadiene styrene (ABS), polyvinyl chlorides (PVCs), polyurethanes, polypropylenes, crosslinkable polymer compositions, polystyrenes, polyethylenes, polyolefins, and co-polymers of at least two polyolefins.

30. The liquid, gas and/or vapor phase producing film of claim 17, wherein the liquid, gas and/or vapor phase producing compound is selected from one or more oxygen gas-producing compounds, nitrogen gas-producing compounds, vapor phase corrosion inhibiting compounds, water, anti-bacterial compounds, anti-viral compounds, anti-static compounds, disinfectants, pain-reliving compounds, anti-coagulant compounds, blood-thinning compounds, blood clotting compounds/promoters, fragrance compounds, stimulants, vitamins, amino-acid supplements, skin-care products, compounds designed to treat acne, odor suppressants, odor enhancing compounds, pharmaceutical compounds, UV-protectant compounds, lubricant compounds, fertilizers, polishing compounds, cleaning compounds, flavor compounds, citrus extracts, medicinal compounds, or compatible mixtures of two or more thereof.

31. The liquid, gas and/or vapor phase producing film of claim 30, wherein the liquid, gas and/or vapor phase producing compound is selected from lithium perchlorate, sodium perchlorate, potassium perchlorate, lithium peroxide, sodium peroxide, potassium peroxide, calcium peroxide, magnesium peroxide, barium peroxide, lead peroxide, carbamide peroxide (CH₆N₂O₃), potassium nitrate, potassium permanganate, chromium (VI) oxide, potassium dichromate, and mixtures of two or more thereof.

32. The liquid, gas and/or vapor phase producing film of claim 30, wherein the liquid, gas and/or vapor phase producing layer further comprises at least one catalyst that facilitates the production of the liquid, gas and/or vapor phase compound from the liquid, gas and/or vapor phase producing layer.

33. The liquid, gas and/or vapor phase producing film of claim 32, wherein the at least one catalyst is selected from sodium permanganate, potassium permanganate, manganese (IV) oxide, or mixtures of two or more thereof.

34. The liquid, gas and/or vapor phase producing film of claim 17, wherein the water-impermeable, gas-permeable layer is formed from a compound selected from polymers, co-polymers, terpolymers, block polymers, block co-polymers, adhesives, and gels.

35. The liquid, gas and/or vapor phase producing film of claim 17, wherein the film is contained within a capsule, bandage, packing, container or enclosure that produces at least one liquid, gas and/or vapor phase compound.

36. The liquid, gas and/or vapor phase producing film of claim 20, wherein the liquid, gas and/or vapor phase producing layer further comprises microcapsules or a combination of microspheres and microcapsules, and wherein the liquid, gas and/or vapor phase producing film further comprises a means to break the microcapsule.

37. The liquid, gas and/or vapor phase producing film of claim 36, wherein the means to break the microcapsules is a sand paper layer positioned between the gas-impermeable and liquid-impermeable layer having and the liquid, gas and/or vapor phase producing layer.

38. The liquid, gas and/or vapor phase producing film of claim 17, further comprising a second active compound layer position between the liquid, gas and/or vapor phase producing layer and the water-impermeable, gas-permeable layer.

39. The liquid, gas and/or vapor phase producing film of claim 38, wherein the second active layer contains at least one compound selected from oxygen gas-producing compounds, nitrogen gas-producing compounds, vapor phase corrosion inhibiting compounds, water, anti-bacterial compounds, anti-viral compounds, anti-static compounds, disinfectants, pain-reliving compounds, anti-coagulant compounds, blood-thinning compounds, blood clotting compounds/promoters, fragrance compounds, stimulants, vitamins, amino-acid supplements, skin-care products, compounds designed to treat acne, odor suppressants, odor enhancing compounds, pharmaceutical compounds, UV-protectant compounds, lubricant compounds, fertilizers, polishing compounds, cleaning compounds, flavor compounds, citrus extracts, medicinal compounds, or compatible mixtures of two or more thereof.

40. The liquid, gas and/or vapor phase producing film of claim 37, further comprising a second active compound layer position between the liquid, gas and/or vapor phase producing layer and the water-impermeable, gas-permeable layer.

41. The liquid, gas and/or vapor phase producing film of claim 40, wherein the second active layer contains at least one compound selected from oxygen gas-producing compounds, nitrogen gas-producing compounds, vapor phase corrosion inhibiting compounds, water, anti-bacterial compounds, anti-viral compounds, anti-static compounds, disinfectants, pain-reliving compounds, anti-coagulant compounds, blood-thinning compounds, blood clotting compounds/promoters, fragrance compounds, stimulants, vitamins, amino-acid supplements, skin-care products, compounds designed to treat acne, odor suppressants, odor enhancing compounds, pharmaceutical compounds, UV-protectant compounds, lubricant compounds, fertilizers, polishing compounds, cleaning compounds, flavor compounds, citrus extracts, medicinal compounds, or compatible mixtures of two or more thereof

42. The liquid, gas and/or vapor phase producing film of claim 17, further comprising a non-stick layer having a first surface and a second surface, the first surface of the non-stick layer facing the second surface of the water-impermeable, gas-permeable layer

43. The liquid, gas and/or vapor phase producing film of claim 37, further comprising a non-stick layer having a first surface and a second surface, the first surface of the non-stick layer facing the second surface of the water-impermeable, gas-permeable layer.

44. A liquid, gas and/or vapor phase producing capsule comprising:
a water-impermeable, gas-permeable layer having a first surface and a second surface;
a liquid, gas and/or vapor phase producing layer having a first surface and a second surface, the first surface of the liquid, gas and/or vapor phase producing layer facing the second surface of the water-impermeable, gas-permeable layer;
a gas funneling or directing layer formed to cover at least a portion of the of the first surface of the water-impermeable, gas-permeable layer;
a gas-impermeable and liquid-impermeable layer surrounding any exposed portion of the water-impermeable, gas-permeable layer and at least the second surface of the liquid, gas and/or vapor phase producing layer,
wherein the liquid, gas and/or vapor phase producing layer contains at least one compound capable of producing a liquid, gas and/or vapor phase compound, and wherein the least one compound that produces the liquid, gas and/or vapor phase compound has a shelf-life of at least about 3 months as a result of being embedded in the liquid, gas and/or vapor phase producing layer.

45. The liquid, gas and/or vapor phase producing capsule of claim 44, wherein the at least one liquid, gas and/or vapor phase producing compound is contained within microspheres, microcapsules, or a combination of microspheres and microcapsules.

46. A liquid, gas and/or vapor phase producing capsule comprising:
a water-impermeable, gas-impermeable shell; and
a one-way vent, the shell defining in conjunction with the one-way vent an interior space, the interior space comprising at least one compound capable of producing a liquid, gas and/or vapor phase compound, wherein the at least one compound that produces the liquid, gas and/or vapor phase compound has a shelf-life of at least about 3 months as a result of being contained within the interior space defined by the combination of the water-impermeable, gas-impermeable shell and the one-way vent.
